(19) United States
(12) Patent Application Publication
(10) Pub. No.: US 2020/0048308 A1
(43) Pub. Date: Feb. 13, 2020

(54) INFLUENZA VIRUS NEUTRALIZING COMPOUNDS

(71) Applicant: JANSSEN VACCINES & PREVENTION B.V., Leiden (NL)

(72) Inventors: Jaroslaw JURASZEK, Amsterdam (NL); Maria VAN DONGEN, Hilversum (NL); Boerries BRANDENBURG, Utrecht (NL)

(21) Appl. No.: 16/344,649

(22) PCT Filed: Oct. 26, 2017
(86) PCT No.: PCT/EP2017/077424

§ 371 Date: Apr. 24, 2019

(30) Foreign Application Priority Data
Oct. 27, 2016 (EP) 16195971.3

(51) Int. Cl. C07K 7/08; C07K 7/64; A61P 31/16

(52) U.S. Cl. CPC C07K 7/08; A61K 38/00; A61P 31/16; C07K 7/64

(57) ABSTRACT

The present invention relates to novel compounds, in particular peptidic macrocyclic peptides, that are capable of binding to and/or neutralizing influenza viruses, in particular influenza A viruses comprising HA of the H1 subtype 1, and to pharmaceutical compositions comprising such compounds. The invention also relates to the use of the peptidomimetic 5 compounds in the diagnosis, prophylaxis and/or treatment of influenza virus infections.

### FIELD OF THE INVENTION

The present invention relates to the field of medicine. The present invention relates to novel compounds that are capable of binding to and/or neutralizing influenza viruses, in particular influenza A viruses comprising HA of the H1 subtype, and to pharmaceutical compositions comprising such compounds. The invention also relates to the use of the influenza virus binding and/or neutralizing compounds in the diagnosis, prophylaxis and/or treatment of influenza virus infections.

### BACKGROUND

Seasonal influenza A is a major public health problem, killing more than 250,000 worldwide each year, while creating an economic burden for millions. Pandemic influenza, which occurs when a new virus emerges and infects people globally that have little or no immunity, represents even a greater threat to human health; for example, the 1918 “Spanish Flu” pandemic caused an estimated 50 million deaths. Of continuing concern is highly pathogenic avian influenza (HPAI) which has demonstrated mortality rates of greater than 50% in infected humans. H5 as well as H7 influenza viruses are endemic in poultry in certain parts of the world. These viruses currently do not appear to be able to transmit readily from person to person, but recent data for avian H5 indicate that only a few amino acid changes are sufficient to enable this virus to spread through aerosol transmission in a mammalian in vivo model system.

Antibodies capable of broadly neutralizing influenza A and/or B viruses have recently been described, such as CR9114 (as disclosed in WO2013/007770), CR6261 (disclosed in WO2008/028946), FI6 (described in Corti et al., Science 333, 850-856 (2011)). These antibodies have been shown to interact with a large variety of hemagglutinin proteins and to neutralize a broad spectrum of influenza strains. As a result of their potency and breadth, such antibodies are now being developed for therapeutic treatment of severely ill patients and prophylactic applications for people belonging to high risk groups. The relative high costs of goods and their parenteral administration, however, are expected to limit the use of monoclonal antibodies in larger populations.

Other currently available agents to prevent and/or treat influenza infection are also associated with severe limitations. Anti-viral drugs such as the neuraminidase inhibitors oseltamivir and zanamivir and the M2 inhibitors amantadine and rimantadine have limited efficacy if administered late in infection and widespread use is likely to result in the emergence of resistant viral strains. Furthermore the use of oseltamivir in adults is associated with adverse effects, such as nausea, vomiting, psychiatric effects and renal events.

Furthermore, the efficacy of influenza vaccines has been shown to be suboptimal for high-risk patients (elderly) and the permanent antigenic changes of the circulating influenza viruses requires annual adaptation of the influenza vaccine formulation to ensure the closest possible match between the influenza vaccine strains and the circulating influenza strains.

The discovery of novel influenza antivirals acting on hemagglutinin (HA) as an alternative strategy to prevent and/or treat influenza infection is also hampered by the large sequence variability of this protein. Hemagglutinin ligands described so far therefore only show activity against a limited number of closely related influenza strains. Recently discovered broadly neutralizing antibodies target a specific epitope on the stem of influenza HA. Binding to this epitope is associated with a low probability of viral escape and increased breadth of binding, solving the most important issues of existing influenza antivirals. However, antibodies are large molecules, and may be difficult and expensive to produce.

In view of the severity of respiratory illness caused by influenza A viruses, as well has the high economic impact of the seasonal epidemics, and the continuing risk for pandemics, there is an ongoing need for new effective inhibitors with broad activity against influenza A viruses and which can be used as medicaments for prevention or treatment of influenza infection.

### SUMMARY OF THE INVENTION

The present invention provides novel compounds, in particular peptidic linear and macrocyclic compounds that are capable of specifically binding to hemagglutinin (HA) of influenza A virus strains comprising HA of the H1 subtype, such as e.g. the H1N1 strains A/California/07/2009 and A/New Caledonia/20/1999. At least some of the compounds of the invention have neutralizing activity against influenza A virus strains comprising HA of the H1 subtype, such as e.g. the H1N1 strains A/California/07/2009 and A/New Caledonia/20/1999. At least some compounds have neutralizing activity against at least two different H1 influenza virus strains.

In certain embodiments, the compounds of the invention comprise the following sequence/formula:

CapN-Tyr-X1-Asp-Pro-X2-Gly-X3-X4-Gly-X5-[Met/Nlu]-CapC

wherein CapN and CapC may be any amino acid sequence comprising from 0-10 residues;

X1 is any charged, hydrophilic or polar L or D-amino acid, as well as any non-canonical hydrophilic, charged or polar L or D amino-acid;

X2 is a hydrophobic, aliphatic or aromatic [canonical or non-canonical] amino acid, provided that X2 is not proline;

X3 is a small or medium aliphatic or hydrophobic L-amino acid, preferably not larger than 150 Da;

X4 is a small aliphatic or hydrophobic L-amino acid, preferably not larger than 100 Da; and

X5 is any polar or charged L- or D-amino acid.

In certain embodiments, the compounds are between 11 and 17 amino acids in length.

In certain embodiments, the compounds have the following sequence/formula:

CapN-Tyr-[Glu/Arg]-Asp-Pro-{Leu/Ph5]-Gly-Val-[Alu/Abu]-Gly-Gly-[Met/Nlu]-CapC

wherein CapN is [Pro|Gly]-Val-Ser-Leu and CapC=Gly-Val-Tyr-D-Pro and CapN and wherein CapN and CapC are linked by a head-to-tail linkage; or

wherein CapN is {Suc}-Val-Ser-Leu and Cap N is Gly-Val-Tyr-{NH2}, wherein CapN and CapC are not connected; or

wherein CapN is D-Pro-Ser-Leu and CapC is Gly-Val-[Pro/Gly] and wherein CapN and CapC are linked by a head-to-tail linkage; or

wherein CapN is [Gly|Pro]-Leu and CapC is Gly-Dsp-Pro and wherein CapN and CapC are linked by a head-to-tail linkage; or

wherein CapN is {Suc}-Cys-Leu and CapC is ly-Cys-{NH2} and wherein CapN and CapC are linked through a cysteine bridge between the Cys residues; or

wherein CapN is Leu and CapC is Gly-Gly and wherein CapN and CapC are linked by a head-to-tail linkage; or

wherein CapN is Leu and CapC is Gly and CapN and CapC are linked by a head-to-tail linkage; or

wherein CapN is {Suc}-Cys and CapC is Cys-{NH2} and CapN and CapC are linked through a cysteine bridge between the cysteine residues.

In certain embodiments, the compound is selected from the group consisting of:

Suc-Cys-Tyr-Glu-Asp-Pro-Leu-Gly-Val-Ala-Gly-Gly-Met-Cys-NH2, which is cyclized through a cysteine bridge (SEQ ID NO: 1);

Suc-Cys-Tyr-Arg-Asp-Pro-Leu-Gly-Val-Ala-Gly-Glu-Met-Cys-NH2, which is cyclized through a cysteine bridge (SEQ ID NO: 2);

Suc-Cys-Tyr-Arg-Asp-Pro-Ph5-Gly-Val-Abu-Gly-Glu-Met-Cys-NH2, which is cyclized through a cysteine bridge (SEQ ID NO: 3);

Suc-Cys-Tyr-Arg-Asp-Pro-Leu-Gly-Val-Ala-Gly-Glu-Nlu-Cys-NH2, which is cyclized through a cysteine bridge (SEQ ID NO: 4);

Leu-Tyr-Glu-Asp-Pro-Leu-Gly-Val-Ala-Gly-Gly-Met-Gly-Gly, which is cyclized through a head-to-tail linkage (SEQ ID NO: 5);

Suc-Cys-Leu-Tyr-Arg-Asp-Pro-Leu-Gly-Val-Ala-Gly-Gly-Met-Gly-Cys-NH2, which is cyclized through a cysteine bridge (SEQ ID NO: 6);

Suc-Cys-Leu-Tyr-Glu-Asp-Pro-Leu-Gly-Val-Ala-Gly-Gly-Met-Gly-Cys-NH2, which is cyclized through a cysteine bridge (SEQ ID NO: 7);

Pro-Leu-Tyr-Glu-Asp-Pro-Leu-Gly-Val-Ala-Gly-Gly-Met-Gly-D-Pro, which is cyclized through a head-to-tail linkage (SEQ ID NO: 8);

Gly-Leu-Tyr-Glu-Asp-Pro-Ph5-Gly-Val-Abu-Gly-Gly-Met-Gly-D-Pro, which is cyclized through a head-to-tail linkage (SEQ ID NO: 9);

Gly-Leu-Tyr-Glu-Asp-Pro-Leu-Gly-Val-Ala-Gly-Gly-Met-Gly-D-Pro, which is cyclized through a head-to-tail linkage (SEQ ID NO: 10);

Suc-Cys-Leu-Tyr-Arg-Asp-Pro-Leu-Gly-Val-Ala-Gly-Glu-Met-Gly-Cys-NH2, which is cyclized through a cysteine bridge (SEQ ID NO: 11);

Suc-Val-Ser-Leu-Tyr-Glu-Asp-Pro-Leu-Gly-Val-Ala-Gly-Gly-Met-Gly-Val-Tyr-NH2 (SEQ ID NO: 12);

Suc-Val-Ser-Leu-Tyr-Arg-Asp-Pro-Leu-Gly-Val-Ala-Gly-Gly-Met-Gly-Val-Tyr-NH2 (SEQ ID NO: 13);

D-Pro-Ser-Leu-Tyr-Glu-Asp-Pro-Leu-Gly-Val-Ala-Gly-Gly-Met-Gly-Val-Pro, which is cyclized through a head-to-tail linkage (SEQ ID NO: 14);

Suc-Val-Ser-Leu-Tyr-Arg-Asp-Pro-Leu-Gly-Val-Ala-Gly-Gly-Nlu-Gly-Val-Tyr-NH2 (SEQ ID NO: 15);

D-Pro-Ser-Leu-Tyr-Glu-Asp-Pro-Leu-Gly-Val-Ala-Gly-Gly-Met-Gly-Val-Gly, which is cyclized through a head-to-tail linkage (SEQ ID NO: 16);

Suc-Val-Ser-Leu-Tyr-Arg-Asp-Pro-Ph5-Gly-Val-Abu-Gly-Glu-Met-Gly-Val-Tyr-NH2 (SEQ ID NO: 17);

Suc-Val-Ser-Leu-Tyr-Arg-Asp-Pro-Leu-Gly-Val-Ala-Gly-Glu-Met-Gly-Val-Tyr-NH2 (SEQ ID NO: 18);

Pro-Val-Ser-Leu-Tyr-Glu-Asp-Pro-Leu-Gly-Val-Ala-Gly-Gly-Met-Gly-Val-Tyr-D-Pro, which is cyclized through a head-to-tail linkage (SEQ ID NO: 19);

Gly-Val-Ser-Leu-Tyr-Glu-Asp-Pro-Leu-Gly-Val-Ala-Gly-Gly-Met-Gly-Val-Tyr-D-Pro, which is cyclized through a head-to-tail linkage (SEQ ID NO: 20);

Leu-Tyr-Glu-Asp-Pro-Leu-Gly-Val-Ala-Gly-Gly-Met-Gly, which is cyclized through a head-to-tail linkage (SEQ ID NO: 21); and

Cys-Tyr-Arg-Asp-Pro-Leu-Gly-Val-Ala-Gly-Gly-Met-Cys, which is cyclized through a cysteine bridge (SEQ ID NO: 22);

The invention furthermore provides pharmaceutical compositions comprising at least one compound as described herein and a pharmaceutically acceptable carrier or diluent.

The invention also relates to compounds as described herein for use in the diagnosis, prevention and/or treatment of influenza.

### DETAILED DESCRIPTION OF THE INVENTION

In the research that led to the present invention novel influenza virus binding and/or neutralizing compounds were developed. In particular, a series of peptidic linear and macrocyclic compounds are provided that mimic the action of the broadly influenza virus neutralizing antibodies, but are preferably only between 11 and 17 amino-acids in length. The compounds of the present invention have been shown to have a competitive binding activity at least towards influenza virus strains comprising HA of the H1 subtype, such as e.g. the H1N1 influenza virus strains A/California/07/2009 and A/New Caledonia/20/1999. At least some of the compounds of the invention also have been shown to have neutralizing activity against influenza A virus strains comprising HA of the H1 subtype, such as e.g. the H1N1 influenza virus strains A/California/07/2009 and A/New Caledonia/20/1999. The compounds of the invention offer several advantages relative to for example anti-hemagglutinin antibodies, including the small size (1.3-1.9 kDa), low cost chemical production, simple engineering into multimeric formats, and high stability with the potential to support non-injectable routes of administration.

In a first aspect, the present invention thus provides compounds comprising the following sequence:

CapN-Tyr-X1-Asp-Pro-X2-Gly-X3-X4-Gly-X5-[Met/Nlu]-CapC

wherein CapN and CapC may be any amino acid sequence comprising from 0-10 residues;

X1 is any charged, hydrophilic or polar L or D-amino acid, as well as any non-canonical hydrophilic, charged or polar L or D amino-acid;

X2 is a hydrophobic, aliphatic or aromatic [canonical or non-canonical] amino acid, provided that X2 is not proline;

X3 is a small or medium aliphatic or hydrophobic L-amino acid, preferably not larger than 150 Da;

X4 is a small aliphatic or hydrophobic L-amino acid preferably not larger than 100 Da; and

X5 is any polar or charged L- or D-amino acid.

The compounds of the invention are based on the CDR3 sequence of the single domain antibody SD1038, which is described in the co-pending patent application WO2016/124768. The sequence of the 20 amino-acid long CDR3 is defined as:

(SEQ ID NO: 23)
Hisl-Va12-Ser3-Leu4-Tyr5-Arg6-Asp7-Pro8-Leu9-
Gly10-Val11-Ala12-Gly13-Gly14-Met15-Gly16-Val17-
Tyr18-Trp19-Gly20.

Using this CDR3 sequence, multiple cyclic peptides have been designed by cyclization in the framework region, as well as by using various linkers.

According to the present invention, X1 thus may be any charged, hydrophilic or polar D or L-amino-acid, such as Arginine, Lysine, Glutamate, Aspartic Acid, Glutamine or Asparagine, as well as any non-canonical hydrophilic, charged or polar L or D amino-acid, such as Ornithine, Citrulline, etc.;

X2 may be any hydrophobic, aliphatic or aromatic amino-acid, canonical or non-canonical, with the proviso however that X2 is not Proline;

X3 may be any small (i.e. <100 Da) or medium (i.e. <150 Da) aliphatic or hydrophobic L-amino-acid, such as Val, Ile, allo-Ile, Abu or Met and their close derivatives;

X4 may be any small (i.e. <100 Da) aliphatic, hydrophobic L-amino-acid, such as Ala, Abu, Trifluoroalanine, 2-Amino-3-butenoic acid, 2-Amino-3-butynoic acid or derivatives thereof; and

X5 may be any polar or charged L- or D-amino-acid.

According to the invention, CapN and CapC may be absent or may be any single amino acid or a chain of 2-10 amino acids, wherein CapN and CapC may be connected by a covalent bond to form a macrocycle by e.g. direct N—C terminus cyclisation, inserting a linker sequence, or a cysteine bridge. Other ways of connecting CapN and CapC are also possible.

In certain embodiments of the invention:

CapN is [Pro|Gly]-Val-Ser-Leu and CapC is Gly-Val-Tyr-D-Pro and CapN, wherein CapN and CapC are linked by a head-to-tail linkage;

CapN is {Suc}-Val-Ser-Leu and Cap N is Gly-Val-Tyr-{NH2}, wherein CapN and CapC are not connected;

CapN is D-Pro-Ser-Leu and CapC is Gly-Val-[Pro/Gly], wherein CapN and CapC are linked by a head-to-tail linkage;

CapN is [Gly|Pro]-Leu and CapC is Gly-Dsp-Pro, wherein CapN and CapC are linked by a head-to-tail linkage;

CapN is {Suc}-Cys-Leu and CapC is ly-Cys-{NH2}, wherein CapN and CapC are linked through a cysteine bridge between the Cys residues in CapN and CapC;

CapN is Leu and CapC is Gly-Gly, wherein CapN and CapC are linked by a head-to-tail linkage;

CapN is Leu and CapC is Gly, wherein CapN and CapC are linked by a head-to-tail linkage; or

CapN is {Suc}-Cys and CapC is Cys-{NH2} and CapN and CapC are linked through a cysteine bridge between the cysteine residues in CapN and CapC.

In certain embodiments, compounds are provided wherein:

X1 is Arginine, Lysine, Glutamate, Aspartic Acid, Glutamine, Asparagine, Ornithine, Citrulline;

X2 is Alanine, Valine, Methionine, Leucine, Isoleucine, Phenylalanine;

X3 is Valine, Isoleucine, allo-Isoleucine, L-2-Aminobutyric acid or Methionine, or close derivatives thereof;

X4 is Alanine, L-2-Aminobutyric acid, Trifluoroalanine, 2-Amino-3-butenoic acid, 2-Amino-3-butynoic acid or derivatives thereof; and

X5 is Glycine, Alanine or Serine.

The compounds of the invention are capable of specifically binding to influenza A virus strain comprising HA of the H1 subtype, such as e.g. the H1N1 influenza virus strains A/California/07/2009 and/or A/New Caledonia/20/1999.

In certain embodiments, the compounds are capable of specifically binding to at least two, preferably to at least three, more preferably to at least four different influenza A virus strains comprising HA of the H1 subtype.

In certain embodiments, the compounds are also capable of neutralizing influenza A virus strains comprising HA of the H1 subtype, such as e.g. the H1N1 influenza virus strains A/California/07/2009 and/or A/New Caledonia/20/1999. In certain embodiment, the compounds are capable of neutralizing at least two, preferably at least three, more preferably at least four different influenza virus strains comprising HA of the H1 subtype

In certain embodiments, the compounds are capable of binding to at least one influenza virus comprising HA of another subtype from phylogenetic group 1, such as the H2, H5 and/or H9 subtype.

The term “specifically binding” as used herein refers to compounds that bind to an epitope of the protein of interest, i.e. HA, but which do not substantially recognize and bind other molecules in a sample containing a mixture of antigenic biological molecules. Typically, the compounds of the invention bind to HA of an influenza A virus of group 1 with an affinity constant (Kd-value) below 10 μM, preferably below 1 μM, more preferably below 0.1 μM, even more preferably below 10 nM, even more preferably below 1 nM.

As used throughout the description, the term “influenza virus subtype” in relation to influenza A viruses refers to influenza A virus strains that are characterized by various combinations of the hemagglutinin (H) and neuraminidase (N) viral surface proteins. Influenza A virus subtypes may be referred to by their H number, such as for example “influenza virus comprising HA of the H1 or H5 subtype”, or “H1 influenza virus”, “H5 influenza virus”, or by a combination of an H number and an N number, such as for example “influenza virus subtype “H1N1” or “H5N1”. The term influenza virus “subtype” specifically includes all individual influenza virus “strains” within such subtype, which usually are different as a result of mutations in hemagglutinin and/or neuraminidase, and show different pathogenic profiles, and include natural isolates as well as man-made mutants or reassortants and the like. Such strains may also be referred to as various “isolates” of a viral subtype. Accordingly, as used herein, the terms “strains” and “isolates” may be used interchangeably. The influenza A virus subtypes can further be classified by reference to their phylogenetic group. Phylogenetic analysis thus has demonstrated a subdivision of influenza hemagglutinins into two main groups: inter alia the H1, H2, H5 and H9 subtypes in phylogenetic group 1 (“group 1” influenza viruses) and inter alia the H3, H4, H7 and H10 subtypes in phylogenetic group 2 (“group 2” influenza viruses).

An amino acid according to the invention can be any of the twenty naturally occurring or variants thereof, such as e.g. D-amino acids (the D-enantiomers of amino acids with a chiral center), or any variants that are not naturally found in proteins. Table 4 shows the abbreviations and properties of the standard amino acids. The 20 amino acids that are encoded directly by the codons of the universal genetic code are called standard or canonical amino acids. The others are called non-standard or non-canonical. Some amino acids have special properties such as e.g. cysteine that can form covalent disulfide bonds to other cysteine residues, proline that forms a cycle to the polypeptide backbone, and glycine that is more flexible than other amino acids.

The term “neutralizing” or “neutralization” as used herein in relation to compounds of the invention refers to the ability of a compound to inhibit an influenza virus from replication, in vitro and/or in vivo within a subject, regardless of the mechanism by which neutralization is achieved. In some embodiments, the compounds of the invention neutralize influenza virus through the inhibition of the fusion of viral and cellular membranes following attachment of the virus to the target cell. The term “cross-neutralizing” or “cross-neutralization” as used herein in relation to the compounds of the invention refers to the ability to neutralize influenza virus strains of different subtypes of influenza A. Neutralizing activity can for instance be measured as described herein. Alternative assays measuring neutralizing activity are described in for instance WHO Manual on Animal Influenza Diagnosis and Surveillance, Geneva: World Health Organisation, 2005, version 2002.5. Typically, the compounds of the invention have a neutralizing activity of 1 μM or less, preferably 100 nM or less, more preferably 10 nM or less, as determined in an in vitro virus neutralization assay (VNA), e.g. as described in the Examples.

In certain embodiments, the compounds of the invention have the following sequence:

CapN-Tyr-[Glu/Arg]-Asp-Pro-lLeu/Ph5]-Gly-Val-[Alu/Abu]-Gly-Gly-[Met/Nlu]-CapC

wherein CapN is [Pro|Gly]-Val-Ser-Leu and CapC=Gly-Val-Tyr-D-Pro and CapN, and wherein CapN and CapC are linked by a head-to-tail linkage;

wherein CapN is {Suc}-Val-Ser-Leu and CapC is Gly-Val-Tyr-{NH2}, wherein CapN and CapC are not connected; or

wherein CapN is D-Pro-Ser-Leu and CapC is Gly-Val-[Pro/Gly] and wherein CapN and CapC are linked by a head-to-tail linkage; or

wherein CapN is [Gly|Pro]-Leu and CapC is Gly-Dsp-Pro and wherein CapN and CapC are linked by a head-to-tail linkage; or

wherein CapN is {Suc}-Cys-Leu and CapC is Gly-Cys-{NH2} and wherein CapN and CapC are linked through a cysteine bridge between the Cys residues in CapN and CapC; or

wherein CapN is Leu and CapC is Gly-Gly and wherein CapN and CapC are linked by a head-to-tail linkage; or

wherein CapN is Leu and CapC is Gly and CapN and CapC are linked by a head-to-tail linkage; or

wherein CapN is {Suc}-Cys and CapC is Cys-{NH2} and CapN and CapC are linked through a cysteine bridge between the cysteine residues in CapN and CapC.

A used throughout this application, Suc=Succinyl; Ph5=(S)-2-Amino-5-phenylpentanoic acid; Abu=L-2-Aminobutyric acid and Nlu=L-Norleucine. The amino acid residues indicated between [ ] in the peptide sequence indicate possible alternative residues, whereas the amino acid residues indicated between { } in the peptide sequence indicate possible alternatives, with a possibility of omitting the amino acid residue.

According to the invention, a “head-to-tail linkage” means formation of the peptide bond between the C and the N terminus of the linear peptide.

In certain embodiments, the compound is selected from the group consisting of:

Suc-Cys-Tyr-Glu-Asp-Pro-Leu-Gly-Val-Ala-Gly-Gly-Met-Cys-NH2, which is cyclized through a cysteine bridge (SEQ ID NO: 1);

Suc-Cys-Tyr-Arg-Asp-Pro-Leu-Gly-Val-Ala-Gly-Glu-Met-Cys-NH2, which is cyclized through a cysteine bridge (SEQ ID NO: 2);

Suc-Cys-Tyr-Arg-Asp-Pro-Ph5-Gly-Val-Abu-Gly-Glu-Met-Cys-NH2, which is cyclized through a cysteine bridge (SEQ ID NO: 3);

Suc-Cys-Tyr-Arg-Asp-Pro-Leu-Gly-Val-Ala-Gly-Glu-Nlu-Cys-NH2, which is cyclized through a cysteine bridge (SEQ ID NO: 4);

Leu-Tyr-Glu-Asp-Pro-Leu-Gly-Val-Ala-Gly-Gly-Met-Gly-Gly, which is cyclized through a head-to-tail linkage (SEQ ID NO: 5);

Suc-Cys-Leu-Tyr-Arg-Asp-Pro-Leu-Gly-Val-Ala-Gly-Gly-Met-Gly-Cys-NH2, which is cyclized through a cysteine bridge (SEQ ID NO: 6);

Suc-Cys-Leu-Tyr-Glu-Asp-Pro-Leu-Gly-Val-Ala-Gly-Gly-Met-Gly-Cys-NH2, which is cyclized through a cysteine bridge (SEQ ID NO: 7);

Pro-Leu-Tyr-Glu-Asp-Pro-Leu-Gly-Val-Ala-Gly-Gly-Met-Gly-D-Pro, which is cyclized through a head-to-tail linkage (SEQ ID NO: 8);

Gly-Leu-Tyr-Glu-Asp-Pro-Ph5-Gly-Val-Abu-Gly-Gly-Met-Gly-D-Pro, which is cyclized through a head-to-tail linkage (SEQ ID NO: 9);

Gly-Leu-Tyr-Glu-Asp-Pro-Leu-Gly-Val-Ala-Gly-Gly-Met-Gly-D-Pro, which is cyclized through a head-to-tail linkage (SEQ ID NO: 10);

Suc-Cys-Leu-Tyr-Arg-Asp-Pro-Leu-Gly-Val-Ala-Gly-Glu-Met-Gly-Cys-NH2, which is cyclized through a cysteine bridge (SEQ ID NO: 11);

Suc-Val-Ser-Leu-Tyr-Glu-Asp-Pro-Leu-Gly-Val-Ala-Gly-Gly-Met-Gly-Val-Tyr-NH2 (SEQ ID NO: 12);

Suc-Val-Ser-Leu-Tyr-Arg-Asp-Pro-Leu-Gly-Val-Ala-Gly-Gly-Met-Gly-Val-Tyr-NH2 (SEQ ID NO: 13);

D-Pro-Ser-Leu-Tyr-Glu-Asp-Pro-Leu-Gly-Val-Ala-Gly-Gly-Met-Gly-Val-Pro, which is cyclized through a head-to-tail linkage (SEQ ID NO: 14);

Suc-Val-Ser-Leu-Tyr-Arg-Asp-Pro-Leu-Gly-Val-Ala-Gly-Gly-Nlu-Gly-Val-Tyr-NH2 (SEQ ID NO: 15);

D-Pro-Ser-Leu-Tyr-Glu-Asp-Pro-Leu-Gly-Val-Ala-Gly-Gly-Met-Gly-Val-Gly, which is cyclized through a head-to-tail linkage (SEQ ID NO: 16);

Suc-Val-Ser-Leu-Tyr-Arg-Asp-Pro-Ph5-Gly-Val-Abu-Gly-Glu-Met-Gly-Val-Tyr-NH2 (SEQ ID NO: 17);

Suc-Val-Ser-Leu-Tyr-Arg-Asp-Pro-Leu-Gly-Val-Ala-Gly-Glu-Met-Gly-Val-Tyr-NH2 (SEQ ID NO: 18);

Pro-Val-Ser-Leu-Tyr-Glu-Asp-Pro-Leu-Gly-Val-Ala-Gly-Gly-Met-Gly-Val-Tyr-D-Pro, which is cyclized through a head-to-tail linkage (SEQ ID NO: 19);

Gly-Val-Ser-Leu-Tyr-Glu-Asp-Pro-Leu-Gly-Val-Ala-Gly-Gly-Met-Gly-Val-Tyr-D-Pro, which is cyclized through a head-to-tail linkage (SEQ ID NO: 20);

Leu-Tyr-Glu-Asp-Pro-Leu-Gly-Val-Ala-Gly-Gly-Met-Gly, which is cyclized through a head-to-tail linkage (SEQ ID NO: 21); and

Cys-Tyr-Arg-Asp-Pro-Leu-Gly-Val-Ala-Gly-Gly-Met-Cys, which is cyclized through a cysteine bridge (SEQ ID NO: 22).

The compounds of the present invention may be prepared by any well know procedure in the art, in particular by the well-established chemical synthesis procedures utilizing automated solid-phase peptide synthesizers followed by chromatographic purification.

The invention further provides pharmaceutical compositions comprising one or more compounds as described herein and a pharmaceutically acceptable carrier or diluent. A “pharmaceutically acceptable excipient” may be any inert substance that is combined with an active molecule such as a compound according to the invention for preparing a suitable composition. The pharmaceutically acceptable excipient is an excipient that is non-toxic to recipients at the used dosages and concentrations, and is compatible with other ingredients of the formulation. Pharmaceutically acceptable excipients are widely applied and known in the art. The pharmaceutical compositions according to the invention may further comprise at least one other therapeutic, prophylactic and/or diagnostic agent. Said further therapeutic and/or prophylactic agents may for example be agents that are also capable of preventing and/or treating an influenza virus infection, such as for example M2 inhibitors (e.g., amantidine, rimantadine) and/or neuraminidase inhibitors (e.g., zanamivir, oseltamivir). These can be used in combination with the compounds of the invention. “In combination” herein means simultaneously, as separate formulations, or as one single combined formulation, or according to a sequential administration regimen as separate formulations, in any order.

In a further aspect, the present invention provides compounds as described herein for use in the diagnosis, prevention and/or treatment of influenza. The invention furthermore provides the use of a compound as described herein in the manufacture of a medicament for the diagnosis, prevention and/or treatment of influenza. As used herein, the term “influenza”, or “influenza virus infection or disease” refers to the pathological condition resulting from an infection of a cell or a subject by an influenza virus. In specific embodiments, the term refers to a respiratory illness caused by an influenza virus. As used herein, the term “influenza virus infection” means the invasion by, multiplication and/or presence of an influenza virus in a cell or a subject. Influenza virus infections can occur in small populations, but can also spread around the world in seasonal epidemics or, worse, in global pandemics where millions of individuals are at risk. The invention provides compounds that can neutralize the infection of influenza strains that cause such seasonal epidemics, as well as potential pandemics. In certain embodiments, the compounds are for use in the diagnosis, prevention and/or treatment of influenza A virus infections, preferably influenza A virus infections caused by an influenza A virus strain from phylogenetic group 1, such as an influenza virus strain comprising HA of the H1 subtype.

The invention further provides methods for preventing and/or treating influenza in a subject, comprising administering a therapeutically effective amount of a compound as described herein to a subject in need thereof. The term “therapeutically effective amount” refers to an amount of the compound as defined herein that is effective for preventing, ameliorating and/or treating a condition resulting from infection with an influenza virus. Prevention and/or treatment may be targeted at patient groups that are susceptible to influenza infection. Such patient groups include, but are not limited to e.g., the elderly (e.g. ≥50 years old, ≥60 years old, and preferably ≥65 years old), the young (e.g. ≤5 years old, ≤1 year old), hospitalized patients and already infected patients who have been treated with an antiviral compound but have shown an inadequate antiviral response.

The compounds of the invention may be administered to a subject for example intravenously, intranasally, via oral inhalation, pulmonary, subcutaneously, intradermally, intravitreally, orally, intramuscularly etc. The optimal route of administration will be influenced by several factors including the physicochemical properties of the active molecules, the urgency of the clinical situation and the relationship of the plasma concentrations of the active molecules to the desired therapeutic effect.

The present invention further provides a method of detecting an influenza A virus in a sample, wherein the method comprises the steps of a) contacting said sample with a diagnostically effective amount of a compound according to the invention, and b) determining whether the compound specifically binds to a molecule in the sample. The sample may be a biological sample including, but not limited to blood, serum, tissue or other biological material from (potentially) infected subjects. The (potentially) infected subjects may be human subjects, but also animals that are suspected as carriers of influenza virus might be tested for the presence of influenza virus using the compounds of the invention.

The present invention is further illustrated in the following, non-limiting Examples.

### EXAMPLES

### Example 1: Synthesis of Compounds of the Invention

A set of 22 compounds of the present invention were designed comprising the general formula:

CapN-Tyr-X1-Asp-Pro-X2-Gly-X3-X4-Gly-X5-[Met|Nlu]-CapC

Exemplary compounds of the invention are listed in the table 1:

TABLE 1
Examplary compounds of the invention

| Molecule | Type of | N- | | C- |
|---|---|---|---|---|
| ID | cyclization | terminus | Sequence (SEQ ID NO:) | terminus |
| CP141088 | head-to-tail | — | LYEDPLGVAGGMG (21) | — |
| CP141068 | cys-bridge | Suc | CYRDPLGVAGGMC (22) | NH2 |
| CP141070 | cys-bridge | Suc | CYEDPLGVAGGMC (1) | NH2 |
| CP141072 | cys-bridge | Suc | CYRDPLGVAGEMC (2) | NH2 |
| CP141093 | cys-bridge | Suc | CYRDP-Ph5-GV-Abu-GEMC (3) | NH2 |
| CP141089 | cys-bridge | Suc | CYRDPLGVAGE-Nlu-C (4) | NH2 |
| CP141087 | head-to-tail | — | LYEDPLGVAGGMGG (5) | — |
| CP141076 | cys-bridge | Suc | CLYRDPLGVAGGMGC (6) | NH2 |
| CP141069 | cys-bridge | Suc | CLYEDPLGVAGGMGC (7) | NH2 |
| CP141086 | head-to-tail | — | PLYEDPLGVAGGMGp (8) | — |
| CP141092 | head-to-tail | — | GLYEDP-Ph5-GV-Abu-GGMGp (9) | — |
| CP141084 | head-to-tail | — | GLYEDPLGVAGGMGp (10) | — |
| CP141071 | cys-bridge | Suc | CLYRDPLGVAGEMGC (11) | NH2 |
| CP141074 | none | Suc | VSLYEDPLGVAGGMGVY (12) | NH2 |
| CP141073 | none | Suc | VSLYRDPLGVAGGMGVY (13) | NH2 |
| CP141091 | head-to-tail | — | pSLYEDPLGVAGGMGVP (14) | — |
| CP141081 | none | Suc | VSLYRDPLGVAGG-Nlu-GVY (15) | NH2 |
| CP141083 | head-tail | — | pSLYEDPLGVAGGMGVG (16) | — |
| CP141090 | none | Suc | VSLYRDP-Ph5-GV-Abu-GEMGVY | |
| | | | (17) | NH2 |
| CP141075 | none | Suc | VSLYRDPLGVAGEMGVY (18) | NH2 |
| CP141085 | head-tail | — | PVSLYEDPLGVAGGMGVYp (19) | — |
| CP141082 | head-tail | — | GVSLYEDPLGVAGGMGVYp (20) | — |

All peptides were prepared by manual solid phase Fmoc peptide chemistry as described below. Amino acid side-chain functionalities were protected as N-Boc (W), O-t-Bu (D,E,S,Y), C-t-Bu (C), and N-Pbf (R) groups (Boc: tert. Butoxycarbonyl, t-Bu: tert. Butyl, Fmoc: 9-Fluorenylmethoxycarbonyl, Pbf: 2,2,4,6,7-pentamethyldihydrobenzofuran-5-sulfonyl).

All chemicals (natural and non-natural amino acids, resins, reagents and solvents) were obtained from commercial suppliers. Purity and identity of the peptides were assessed by HPLC and mass spectrometry.

### Linear Peptides

Linear peptides were prepared by manual solid phase Fmoc chemistry on a Rink amide MBHA resin (0.53 mmol/g). The resin was swelled in DMF (dimethylformamide) for 1 hour and treated with 20% piperidine in DMF (2×15 min) to effect Fmoc removal. All acylation reactions were carried out using a 3-fold excess of Fmoc-amino acid activated with 0.95 eq. of HBTU ((2-(1H-benzotriazole-1-yl)-1,1,3,3-tetramethyluronium hexafluorophosphate) in presence of 2 eq. of DIPEA (N,N-diisopropylethylamine), a coupling time of 1.5 à 2 hours was used. N-terminal succinylation was performed by treatment of the peptide-resin with succinic anhydride (5 eq.) in presence of DIPEA (10 eq.) (1 hour).

Peptides were cleaved from the resin and deprotected using trifluoroacetic acid/thioanisole/1,2-ethanedithiol/water (90:5:2.5:2.5) for 3 hours. The resin was removed by filtration, after which the filtrate was poured into ice-cold tert-butyl methyl ether resulting in the precipitation of the crude peptide. Final purification was done by RP-HPLC (reversed-phase high performance liquid chromatography).

### Disulfide Cyclized Peptides

Disulfide cyclization was effected by iodine oxidation. The crude linear peptide precursor, prepared as described above, was dissolved in water/acetonitrile (7:3) and an iodine solution (0.1 M in methanol) was added until permanent discoloration of the reaction mixture was observed. Subsequent lyophilization afforded the crude cyclized peptide which was purified by RP-HPLC.

### Head to Tail Lactam Cyclized Peptides

Linear peptides were prepared by manual solid phase Fmoc chemistry on a glycine preloaded 2-chlorotrityl chloride resin (0.5 mmol/g) according to the protocol described above. Peptides were cleaved from the resin by swirling the peptide-resin for one hour in a mixture of dichloromethane/hexafluoroisopropanol/trifluoroethanol/triisopropylsilane (6.5:2:1:0.5). The resin was filtered off, and the peptide was precipitated from the filtrate by the addition of cold tert-butyl methyl ether. The peptide was dried under vacuum and used as such in the subsequent lactam cyclization step.

Lactam cyclization was performed at high dilution by dissolving the linear side chain protected peptide in DMF (0.001 M), to which a solution of PyBOP (benzotriazol-1-yl-oxytripyrrolidinophosphonium hexafluorophosphate, 2 eq., 0.004 M) and N-methylmorpholine (6 eq., 0.012 M) in DMF was added dropwise. The reaction mixture was stirred at room temperature until complete conversion was observed. Removal of the solvent under reduced pressure afforded the cyclized peptide which was completely deprotected by treatment with a mixture of using trifluoroacetic acid/thioanisole/1,2-ethanedithiol/water (90:5:2.5:2.5) at room temperature for three hours. The reaction mixture was poured into ice-cold tert-butyl methyl ether resulting in the precipitation of the crude peptide. Final purification was done by RP-HPLC.

High Performance Liquid Chromatography (HPLC) was performed on an Agilent 1200 HPLC system using a ZORBAX Eclipse XDB-C18 column (5 μm, 4.6×150 mm) with a flow rate of 0.8 mL/min and a column temperature of 30° C. A linear AB gradient of 2% B/min was applied starting at 10% B to 50% B, followed by a linear AB gradient of 2.6% B/min to 90% B (solvent A: 0.1% TFA in water, solvent B: 0.05% TFA in ACN). Detection was done using a diode-array (DAD) and Mass Selective Detector (MSD). Peptide masses were calculated from the experimental mass to charge (m/z) ratios.

### Example 2: Binding of Compounds to Influenza HA and Competition of Compounds with Other HA Binders

Binding competition studies were designed to test compounds of the invention for competition with other well characterized HA binding proteins (such as e.g. CR9114, CR6261 and HB80.4) with known epitopes on HA. The epitopes where either located at the stem of the HA (viral membrane proximal part of HA) or, for control purposes, at the head of HA (viral membrane distal part of HA). If competition was observed, it was assumed that both molecules bind to a similar or at least overlapping epitope at the surface of HA. Competition with a HA head- and stem-binder was interpreted as unspecific binding.

Hereto an AlphaLISA competition assay (Perkin Elmer) was established which relied on biotinylated full length and trimeric HA proteins (Protein Sciences, 10 μL, 0.5 nM final concentration in 50 μL) bound by HA-specific binders. The interaction between HA and the binder was detected after 1 h incubation at room temperature (RT) with two beads, a streptavidin donor bead recognizing HA (10 μL of 10 μg/mL) and an anti Fc bead (10 μg/mL) recognizing the IgGs used. If after an additional hour of incubation at RT the excited donor bead (680 nm) and acceptor bead are in close proximity, an energy transfer (singlet oxygen) can be measured as a luminescence signal of the acceptor bead (Perkin Elmer EnVision plate reader). The signal intensity in this homogeneous assay format is directly proportional to the binding strength (affinity/avidity) between both binding partners. A competitor (compound), depending on its affinity and concentration (usually tested in a range from 100 nM to 0.5 pM) can disrupt the AlphaLISA signal leading to a sigmoidal inhibitor curve which is fitted with a standard four parameter logistic nonlinear regression model in SPSS. Calculated pIC50 values are shown in Table 2.

TABLE 2
Calculated pIC50 values

| ID | ALC_H1_Cal_HB80.4 | ALC_H1_NCa_HB80.4 |
|---|---|---|
| CP141085 | 7.8 | 7.9 |
| CP141082 | 7.7 | 7.7 |
| CP141076 | 7.0 | 6.9 |
| CP141088 | 6.6 | 6.6 |
| CP141069 | 6.5 | 6.8 |
| CP141092 | 6.4 | 6.6 |
| CP141071 | 6.3 | 5.7 |
| CP141087 | 6.3 | 6.2 |
| CP141068 | 6.1 | 5.4 |
| CP141084 | 6.1 | 6.4 |
| CP141086 | 5.8 | 6.7 |
| CP141073 | 5.7 | 5.2 |
| CP141081 | 5.5 | 5.1 |
| CP141074 | 5.4 | 5.3 |
| CP141083 | 5.3 | 5.0 |
| CP141091 | 5.3 | 5.2 |
| CP141070 | 5.2 | 5.4 |
| CP141093 | 5.0 | 4.7 |
| CP141075 | 5.0 | 4.3 |
| CP141090 | 5.0 | 4.8 |
| CP141072 | 4.8 | 4.9 |
| CP141089 | 4.6 | 4.3 |

The compounds of the invention bind to HA as shown through competition with well-known binding molecules. In the alphalisa assay, the best compound—CP141085—reached IC50 of 13 nM. The least potent peptides competed with an IC50 in the 10 μM range. Activity has been demonstrated on two H1 strains, H1/Cal and H1/Nca, which represent the two most abundant H1 stem epitopes and covered 60% of all stem epitope sequence variation as of 2011, based on sequences available in the NCBI flu database. In conclusion, the compounds of the invention bind to HA as shown through competition with well-known HA binding molecules in the range from 10 μM to close to 10 nM.

### Example 3: Influenza Virus Neutralizing Activity and Cell Toxicity of Compounds

Compounds were analyzed in a virus neutralization assay (VNA) for their ability to prevent influenza virus infection of mammalian cells. For this purpose, human lung epithelia derived Calu-3 cells (ATCC, cat # HTB-55) were seeded in 96-well plates (4E+04 cells/well) and incubated for at least 7 days at 37° C. and 10% CO2 in complete DMEM (containing 1×MEM Non-Essential Amino Acids, 2 mM L-Glutamine, and 10% FBSHI origin: Australia; Gibco). Polarized Calu-3 cells at about 90% confluency and established tight junctions are ready for the VNA. On the day of the assay, sample dilutions plates were prepared from compound stock (dissolved in PBS 0.1% BSA, 0.1% Tween 5% DMSO, 500 nM start concentration) 2 fold diluted in incomplete DMEM (containing 2 mM L-glutamine, 1×pen/strep). Sample dilution plates were centrifuged (1000 g for 15 min) to remove potential aggregates. 5 TCID50/50 μL influenza virus (pre-titrated on Calu-3 cells) in incomplete DMEM was then added to the sample dilution plate and incubated for 1 hour at 37° C. and 10% CO2. The medium was removed from the cells and replaced with 50 μL incomplete DMEM supplemented with 3% FBS. 100 μL Virus/compound mix was then added to the cells resulting in a total assay volume of 150 μL with a final concentration of 1% FBS. After incubating for 4 days at 37° C. and 10% CO2 cells were washed with PBS and fixed with 200 μL/well 80% Acetone for 15 min at room temperature (RT). The level of influenza infection was determined influenza nucleoprotein (NP) ELISA. The primary antibody anti-Flu-A-NP (Abbiotec, Clone 5D8) was used at 1:1000 diluted in 1% BSA in PBS and incubated for 1 hour shaking at 300 rpm at RT. After washing the cells three times with wash-buffer (PBS, 0.05% Tween), the secondary antibody (anti-Mouse HRP, 1:2000) was added and incubated for 1 hour shaking at 300 rpm at RT. After washing the cells three times, 50 μL/well POD chemiluminescence substrate was added and incubated for 2-5 min before reading luminescence on the Biotek Synergy Neo Plate Reader. The pIC50 of compounds was calculated with the SPSS software. Results are presented in the table 3:

TABLE 3
pIC50 values

| ID | VNA_H1_NCa_Calu3 |
|---|---|
| CP141085 | 5.9 |
| CP141082 | 4.3 |
| CP141076 | 6.0 |
| CP141088 | 4.4 |
| CP141069 | 6.1 |
| CP141092 | 4.0 |
| CP141071 | 4.0 |
| CP141087 | 4.9 |
| CP141068 | 4.0 |
| CP141084 | 5.7 |
| CP141086 | 4.6 |
| CP141073 | 4.0 |
| CP141081 | 4.0 |
| CP141074 | 4.0 |
| CP141083 | 4.0 |
| CP141091 | 4.2 |
| CP141070 | 4.4 |
| CP141093 | 4.0 |
| CP141075 | 4.0 |
| CP141090 | 4.0 |
| CP141072 | 4.0 |
| CP141089 | 4.0 |

In conclusion, some compounds of the invention reach micromolar neutralizing activity against A/New Caledonia, indicating increased proteolytic stability.

TABLE 4
Standard amino acids, abbreviations and properties

| | | | Side chain | Side chain |
|---|---|---|---|---|
| Amino Acid | 3-Letter | 1-Letter | polarity | charge (pH 7.4) |
| alanine | Ala | A | non-polar | Neutral |
| arginine | Arg | R | polar | Positive |
| asparagine | Asn | N | polar | Neutral |
| aspartic acid | Asp | D | polar | Negative |
| cysteine | Cys | C | non-polar | Neutral |
| glutamic acid | Glu | E | polar | Negative |
| glutamine | Gln | Q | polar | Neutral |
| glycine | Gly | G | non-polar | Neutral |
| histidine | His | H | polar | Positive (10%) |
| | | | | neutral (90%) |
| isoleucine | Ile | I | non-polar | Neutral |
| leucine | Leu | L | non-polar | Neutral |
| lysine | Lys | K | polar | Positive |
| methionine | Met | M | non-polar | Neutral |
| phenylalanine | Phe | F | non-polar | Neutral |
| proline | Pro | P | non-polar | Neutral |
| serine | Ser | S | polar | Neutral |
| threonine | Thr | T | polar | Neutral |
| tryptophan | Trp | W | non-polar | Neutral |
| tyrosine | Tyr | Y | polar | Neutral |
| valine | Val | V | non-polar | Neutral |

1. A compound comprising the sequence:
CapN-Tyr-X1-Asp-Pro-X2-Gly-X3-X4-Gly-X5-[Met/Nlu]-CapC,
wherein CapN and CapC each are an amino acid sequence comprising from 0-10 residues;
X1 is any charged, hydrophilic or polar L or D-amino acid, as well as any non-canonical hydrophilic, charged or polar L or D amino-acid;
X2 is a hydrophobic, aliphatic or aromatic, canonical or non-canonical amino acid, provided that X2 is not proline;
X3 is a small or medium aliphatic or hydrophobic L-amino acid;
X4 is a small aliphatic or hydrophobic L-amino acid; and
X5 is any polar or charged L- or D-amino acid,
and wherein the compound is capable of specifically binding to hemagglutinin (HA) of an influenza A virus strain comprising HA of the H1 subtype.

2. The compound according to claim 1, which is furthermore capable of neutralizing an influenza A virus strain comprising HA of the H1 subtype.

3. The compound according to claim 2, wherein the influenza A virus strains comprising HA of the H1 subtype is the H1N1 influenza virus strain A/California/07/2009 or A/New Caledonia/20/1999.

4. The compound according to claim 1, wherein:
X1 is Arginine, Lysine, Glutamate, Aspartic Acid, Glutamine, Asparagine, Ornithine, Citrulline;
X2 is Alanine, Valine, Methionine, Leucine, Isoleucine, Phenylalanine;
X3 is Valine, Isoleucine, allo-Isoleucine, L-2-Aminobutyric acid or Methionine, or close derivatives thereof;
X4 is Alanine, L-2-Aminobutyric acid, Trifluoroalanine, 2-Amino-3-butenoic acid, 2-Amino-3-butynoic acid or derivatives thereof; and
X5 is Glycine, Alanine or Serine.

5. The compound according to claim 1, wherein:
CapN is [Pro|Gly]-Val-Ser-Leu and CapC is Gly-Val-Tyr-D-Pro and CapN, and wherein CapN and CapC are linked by a head-to-tail linkage;
CapN is {Suc}-Val-Ser-Leu and CapC is Gly-Val-Tyr-{NH2}, wherein CapN and CapC are not connected;
CapN is D-Pro-Ser-Leu and CapC is Gly-Val-[Pro/Gly] and wherein CapN and CapC are linked by a head-to-tail linkage;
CapN is [Gly|Pro]-Leu and CapC is Gly-Dsp-Pro and wherein CapN and CapC are linked by a head-to-tail linkage;
CapN is {Suc}-Cys-Leu and CapC is ly-Cys-{NH2} and wherein CapN and CapC are linked through a cysteine bridge between the Cys residues in CapN and CapC;
CapN is Leu and CapC is Gly-Gly and wherein CapN and CapC are linked by a head-to-tail linkage;
CapN is Leu and CapC is Gly and CapN and CapC are linked by a head-to-tail linkage; or
CapN is {Suc}-Cys and CapC is Cys-{NH2} and CapN and CapC are linked through a cysteine bridge between the cysteine residues in CapN and CapC.

6. The compound according to claim 1, comprising the sequence:
CapN-Tyr-[Glu/Arg]-Asp-Pro-lLeu/Ph5]-Gly-Val-[Alu/Abu]-Gly-Gly-[Met/Nlu]-CapC.

7. A compound selected from the group consisting of:
Suc-Cys-Tyr-Glu-Asp-Pro-Leu-Gly-Val-Ala-Gly-Gly-Met-Cys-NH2, which is cyclized through a cysteine bridge (SEQ ID NO: 1);
Suc-Cys-Tyr-Arg-Asp-Pro-Leu-Gly-Val-Ala-Gly-Glu-Met-Cys-NH2, which is cyclized through a cysteine bridge (SEQ ID NO: 2);
Suc-Cys-Tyr-Arg-Asp-Pro-Ph5-Gly-Val-Abu-Gly-Glu-Met-Cys-NH2, which is cyclized through a cysteine bridge (SEQ ID NO: 3);
Suc-Cys-Tyr-Arg-Asp-Pro-Leu-Gly-Val-Ala-Gly-Glu-Nlu-Cys-NH2, which is cyclized through a cysteine bridge (SEQ ID NO: 4);
Leu-Tyr-Glu-Asp-Pro-Leu-Gly-Val-Ala-Gly-Gly-Met-Gly-Gly, which is cyclized through a head-to-tail linkage (SEQ ID NO: 5);
Suc-Cys-Leu-Tyr-Arg-Asp-Pro-Leu-Gly-Val-Ala-Gly-Gly-Met-Gly-Cys-NH2, which is cyclized through a cysteine bridge (SEQ ID NO: 6);
Suc-Cys-Leu-Tyr-Glu-Asp-Pro-Leu-Gly-Val-Ala-Gly-Gly-Met-Gly-Cys-NH2, which is cyclized through a cysteine bridge (SEQ ID NO: 7);
Pro-Leu-Tyr-Glu-Asp-Pro-Leu-Gly-Val-Ala-Gly-Gly-Met-Gly-D-Pro, which is cyclized through a head-to-tail linkage (SEQ ID NO: 8);
Gly-Leu-Tyr-Glu-Asp-Pro-Ph5-Gly-Val-Abu-Gly-Gly-Met-Gly-D-Pro, which is cyclized through a head-to-tail linkage (SEQ ID NO: 9);
Gly-Leu-Tyr-Glu-Asp-Pro-Leu-Gly-Val-Ala-Gly-Gly-Met-Gly-D-Pro, which is cyclized through a head-to-tail linkage (SEQ ID NO: 10);
Suc-Cys-Leu-Tyr-Arg-Asp-Pro-Leu-Gly-Val-Ala-Gly-Glu-Met-Gly-Cys-NH2, which is cyclized through a cysteine bridge (SEQ ID NO: 11);
Suc-Val-Ser-Leu-Tyr-Glu-Asp-Pro-Leu-Gly-Val-Ala-Gly-Gly-Met-Gly-Val-Tyr-NH2 (SEQ ID NO: 12);
Suc-Val-Ser-Leu-Tyr-Arg-Asp-Pro-Leu-Gly-Val-Ala-Gly-Gly-Met-Gly-Val-Tyr-NH2 (SEQ ID NO: 13);
D-Pro-Ser-Leu-Tyr-Glu-Asp-Pro-Leu-Gly-Val-Ala-Gly-Gly-Met-Gly-Val-Pro, which is cyclized through a head-to-tail linkage (SEQ ID NO: 14);
Suc-Val-Ser-Leu-Tyr-Arg-Asp-Pro-Leu-Gly-Val-Ala-Gly-Gly-Nlu-Gly-Val-Tyr-NH2 (SEQ ID NO: 15);
D-Pro-Ser-Leu-Tyr-Glu-Asp-Pro-Leu-Gly-Val-Ala-Gly-Gly-Met-Gly-Val-Gly, which is cyclized through a head-to-tail linkage (SEQ ID NO: 16);
Suc-Val-Ser-Leu-Tyr-Arg-Asp-Pro-Ph5-Gly-Val-Abu-Gly-Glu-Met-Gly-Val-Tyr-NH2 (SEQ ID NO: 17);
Suc-Val-Ser-Leu-Tyr-Arg-Asp-Pro-Leu-Gly-Val-Ala-Gly-Glu-Met-Gly-Val-Tyr-NH2 (SEQ ID NO: 18);
Pro-Val-Ser-Leu-Tyr-Glu-Asp-Pro-Leu-Gly-Val-Ala-Gly-Gly-Met-Gly-Val-Tyr-D-Pro, which is cyclized through a head-to-tail linkage (SEQ ID NO: 19);
Gly-Val-Ser-Leu-Tyr-Glu-Asp-Pro-Leu-Gly-Val-Ala-Gly-Gly-Met-Gly-Val-Tyr-D-Pro, which is cyclized through a head-to-tail linkage (SEQ ID NO: 20);
Leu-Tyr-Glu-Asp-Pro-Leu-Gly-Val-Ala-Gly-Gly-Met-Gly, which is cyclized through a head-to-tail linkage (SEQ ID NO: 21); and
Cys-Tyr-Arg-Asp-Pro-Leu-Gly-Val-Ala-Gly-Gly-Met-Cys, which is cyclized through a cysteine bridge (SEQ ID NO: 22).

8. A pharmaceutical composition comprising a compound according to claim 1 and a pharmaceutically acceptable carrier or diluent.

9. The compound according to claim 1 for use in the diagnosis, prevention and/or treatment of influenza.

10. (canceled)

11. The compound according to claim 5, comprising the sequence:
CapN-Tyr-[Glu/Arg]-Asp-Pro-lLeu/Ph5]-Gly-Val-[Alu/Abu]-Gly-Gly-[Met/Nlu]-CapC,
wherein CapN and CapC are as defined in claim 5.

12. A pharmaceutical composition comprising a compound according to claim 5 and a pharmaceutically acceptable carrier or diluent.

13. A pharmaceutical composition comprising a compound according to claim 7 and a pharmaceutically acceptable carrier or diluent.

14. A method for the diagnosis, prophylaxis, and/or treatment of influenza in a subject in need thereof, comprising administering a therapeutically effective amount of a compound as defined in claim 1 to the subject.

15. A method for the diagnosis, prophylaxis, and/or treatment of influenza in a subject in need thereof, comprising administering a therapeutically effective amount of a compound as defined in claim 5 to the subject.

16. A method for the diagnosis, prophylaxis, and/or treatment of influenza in a subject in need thereof, comprising administering a therapeutically effective amount of a compound as defined in claim 7 to the subject.
